(19) United States
(12) Patent Application Publication
(10) Pub. No.: US 2011/0217253 A1
(43) Pub. Date: Sep. 8, 2011

(54) COMPOSITION CONTAINING A BLOCK COPOLYMER AND A SPECFIC NON-VOLATILE OIL

(75) Inventors: Pascal Arnaud, L'Hay Les Roses (FR); Camille Thibout, Paris (FR)

(73) Assignee: L'OREAL, Paris (FR)

(21) Appl. No.: 12/876,395

(22) Filed: Sep. 7, 2010

Related U.S. Application Data
(60) Provisional application No. 61/242,046, filed on Sep. 14, 2009.

(30) Foreign Application Priority Data
Sep. 8, 2009 (FR) 09 56108

(51) Int. Cl. A61K 8/90; A61Q 1/02

(52) U.S. Cl. 424/63

(57) ABSTRACT

Cosmetic care and/or makeup composition containing a particular block ethylenic copolymer, a hydrocarbon-based non-volatile oil characterized by a Hansen solubility parameter δa ranging from 2 to 7 (J/cm³)^{1/2} and a molecular mass of less than or equal to 300 g/mol, and a volatile oil.

### REFERENCE TO PRIOR APPLICATIONS

This application claims priority to U.S. provisional application Ser. No. 61/242,046, filed Sep. 14, 2009; and to French patent application 09 56108, filed Sep. 8, 2009, both incorporated herein by reference.

### SUMMARY OF THE INVENTION

The present invention relates to care and/or makeup compositions for keratin materials, in particular in the form of W/O emulsions, which have good staying power and advantageously comfort properties after application to the keratin fibres, in particular the skin.

### BACKGROUND OF THE INVENTION

When women use a cosmetic product such as a makeup product, an antisun product or alternatively a care product, they want this product to have good staying power after application.

It is known to those skilled in the art to use polymers for obtaining these properties of staying power during the day.

These polymers are very different in chemical nature and are carried either in a fatty phase or in an aqueous phase.

By way of examples, mention may be made of silicone resins, polyacrylates, latices etc.

These polymers are often combined with volatile compounds which make it possible to improve the staying power and non-volatile oils which promote comfort.

However, if these non-volatile oils are not chosen appropriately with respect to the polymer, they can be prejudicial to the properties of staying power of the composition and even of comfort of said composition.

This is because incompatibility between these oils and the polymer in the composition can reduce its effectiveness with respect to staying power and/or make it sticky. They can also solubilize it to too great an extent, thereby preventing it from forming a film after application and here again reducing its influence on the staying power.

It therefore remains necessary to find optimum combinations between polymers and non-volatile oils.

### DETAILED DESCRIPTION OF THE PREFERRED EMBODIMENTS

The inventors have found, unexpectedly, that the combination of a block copolymer as defined hereinafter, with certain non-volatile oils and volatile oils, makes it possible to obtain optimized staying power properties and with comfort properties.

While not bound by theory, it is thought that, like the polymer, the volatile oil makes it possible to improve the staying power properties of the composition. It may be a hydrocarbon-based, silicone or else fluoro oil, preferably a hydrocarbon-based oil. It can also constitute a medium for carrying the polymer before it is introduced into the composition.

The preferred non-volatile oil of the invention is hydrocarbon-based and it is characterized by a Hansen solubility parameter δa of between 2 and 7 (J/cm²)^{1/2} and a molecular mass of less than or equal to 300 g/mol. Without being bound by any theory, when the non-volatile oil has a δa solubility parameter outside this range, it is not compatible enough with the polymer and is unfavourable to the properties of staying power provided by the polymer.

Preferably, the ratio of the polymer to the non-volatile oil is between 0.5 and 100, and even more preferably between 1 and 40.

The use of copolymers as described hereinafter in the present invention, in cosmetic care or makeup compositions, in particular lipsticks having good staying power properties, is known from the prior art (EP 1 411 069 from L'Oréal or EP 1 518 535 from L'Oréal both incorporated herein by reference).

However, to the applicant's knowledge, combining them specifically with hydrocarbon-based non-volatile oils characterized by a Hansen solubility parameter δa ranging from 2 to 7 (J/cm³)^{1/2} and a molecular mass of less than or equal to 300 g/mol, in order to obtain products having improved properties in terms of staying power and comfort after application, has never been described.

The invention therefore includes a cosmetic care or makeup composition for keratin materials, comprising at least a liquid fatty phase and at least:
a) a block ethylenic copolymer (also referred to herein as a block ethylenic polymer) containing at least a first block having a glass transition temperature (Tg) of greater than or equal to 40° C. and being completely or partially derived from one or more first monomers, which are such that the homopolymer prepared from these monomers has a glass transition temperature of greater than or equal to 40° C., and at least one second block having a glass transition temperature of less than or equal to 20° C. and being completely or partially derived from one or more second monomers, which are such that the homopolymer prepared from these monomers has a glass transition temperature of less than or equal to 20° C., said first block and said second block being linked together via a random intermediate segment comprising at least one of said first constituent monomers of the first block and at least one of said second constituent monomers of the second block, and said block copolymer having a polydispersity index I of greater than 2,
b) a hydrocarbon-based non-volatile oil characterized by a Hansen solubility parameter δa ranging from 2 to 7 (J/cm³)^{1/2} and a molecular mass of less than or equal to 300 g/mol, and
c) a volatile oil.

Preferably, the block copolymer (a) and the non-volatile oil (b) are present in said composition in contents such that the (a)/(b) weight ratio ranges from 0.5 to 100, preferably from 1 to 40.

Preferably, said composition is in the form of an emulsion, preferably a water-in-oil (W/O) emulsion.

According to one particular mode, it is a foundation.

The combination according to the invention also makes it possible to formulate compositions in the form of dispersions or emulsions without silicone compounds.

The invention also relates to a cosmetic care or makeup process for keratin materials, comprising the application to said keratin materials, and in particular the skin, of a composition according to the invention.

Block Copolymers

The composition of the invention comprises at least one block copolymer.

These polymers comprise at least one first block and at least one second block, which are incompatible with one another, and are linked via an intermediate block which comprises at least one constituent monomer of each of said two blocks. These monomers are preferably of alkyl acrylate or methacrylate type, in particular methyl acrylate or methyl methacrylate, isobutyl acrylate or methacrylate, isobornyl acrylate or methacrylate or (meth)acrylic acid type.

These polymers can generally be conveyed in volatile or non-volatile solvents, in particular alkanes such as isododecane.

In this family, mention may preferentially be made of the poly(isobornyl acrylate/isobornyl methacrylate/isobutyl acrylate/acrylic acid) copolymer, the synthesis of which is described in Example 1 described hereinafter.

According to the invention, the block copolymer may be present in a content with respect to active material ranging from 0.1% to 30%, preferably between 0.5% and 20%, and even more preferably between 1% and 10% by weight of active material of block copolymer relative to the total weight of said composition.

The block polymers (copolymers) that can be used according to the invention are, for example, those described in Patent Application EP 1 411 069 A2 from L'Oréal.

These copolymers comprise a first and at least one second block, said first and second blocks being linked via an intermediate block which comprises at least one constituent monomer of each of said two blocks.

The composition according to the invention therefore comprises at least one block ethylenic copolymer (also referred to as block ethylenic polymer) containing at least one first block having a glass transition temperature (Tg) of greater than or equal to 40° C. and being completely or partially derived from one or more first monomers, which are such that the homopolymer prepared from these monomers has a glass transition temperature of greater than or equal to 40° C., and at least one second block having a glass transition temperature of less than or equal to 20° C. and being completely or partially derived from one or more second monomers, which are such that the homopolymer prepared from these monomers has a glass transition temperature of less than or equal to 20° C., said first block and said second block being linked together via a random intermediate segment comprising at least one of said first constituent monomers of the first block and at least one of said second constituent monomers of the second block, and said block copolymer having a polydispersity index I of greater than 2.

The block polymer used in the invention thus comprises at least one first block and at least one second block.

The term “at least” one block is intended to mean one or more blocks.

The term “block” polymer is intended to mean a polymer comprising at least 2 distinct blocks, preferably at least 3 distinct blocks.

The term “ethylenic” polymer is intended to mean a polymer obtained by polymerization of monomers comprising an ethylenic unsaturation.

The block ethylenic polymer used according to the invention is prepared exclusively from monofunctional monomers.

This means that the block ethylenic polymer used according to the present invention does not contain multifunctional monomers, which make it possible to break the linearity of a polymer in order to obtain a branched or a crosslinked polymer, according to the degree of multifunctional monomer. The polymer used according to the invention also does not contain macromonomers (the term “macromonomer” is intended to mean a monofunctional monomer having a pendent group of polymeric nature, and preferably having a molecular mass of greater than 500 g/mol, or else a polymer comprising, on just one of its ends, an end group which is polymerizable (or comprising an ethylenic unsaturation)), which are used for the preparation of a grafted polymer.

It is specified that, in the above text and in the text which follows, the terms “first” and “second” blocks do not in any way condition the order of said blocks in the structure of the polymer.

The first block and the second block of the polymer used in the invention can be advantageously incompatible with one another.

The expression “blocks incompatible with one another” is intended to mean that the blend formed by a polymer corresponding to the first block and by a polymer corresponding to the second block is not miscible in the polymerization solvent, which is predominant by weight, of the block polymer, at ambient temperature (25° C.) and atmospheric pressure (10⁵ Pa) for a content of the blend of said polymers of greater than or equal to 5% by weight, relative to the total weight of the blend of said polymers and of said polymerization solvent, it being understood that:
i) said polymers are present in the blend in a content such that the respective weight ratio ranges from 10/90 to 90/10, and that
ii) each of the polymers corresponding to the first and second blocks has an average molecular mass (weight-average or number-average) equal to that of the block polymer+/−15%.

In the case of a mixture of polymerization solvents, in the event of there being two or more solvents present in identical proportions by mass, said blend of polymers is immiscible in at least one of them.

Of course, in the case of a polymerization carried out in a single solvent, the latter is the predominant solvent.

The block polymer according to the invention comprises at least one first block and at least one second block linked together via an intermediate segment comprising at least one constitutive monomer of the first block and at least one constitutive monomer of the second block. The intermediate segment (also referred to as intermediate block) has a glass transition temperature Tg between the glass transition temperatures of the first and second blocks.

The intermediate segment is a block comprising at least one constitutive monomer of the first block and at least one constitutive monomer of the second block of the polymer, and makes it possible to “compatibilize” these blocks.

The intermediate segment comprising at least one constitutive monomer of the first block and at least one constitutive monomer of the second block of the polymer is a random polymer.

Preferably, the intermediate block is derived essentially from constitutive monomers of the first block and of the second block.

The term “essentially” is intended to mean at least to 85%, preferably at least to 90%, better still to 95% and even better still to 100%.

The block polymer according to the invention is advantageously a film-forming block ethylenic polymer.

The term “ethylenic” polymer is intended to mean a polymer obtained by polymerization of monomers comprising an ethylenic unsaturation.

The term “film-forming” polymer is intended to mean a polymer capable of forming, on its own or in the presence of an auxiliary film-forming agent, a continuous deposit on a substrate, in particular on keratin materials.

Preferably, the polymer according to the invention does not comprise silicon atoms in its backbone. The term “backbone” is intended to mean the main chain of the polymer, as opposed to the pendent side chains.

Preferably, the polymer according to the invention is not water-soluble, i.e. the polymer is not soluble in water or in a mixture of water and linear or branched lower monoalcohols containing from 2 to 5 carbon atoms, such as ethanol, isopropanol or n-propanol, without modification of pH, at a content of active material of at least 1% by weight, at ambient temperature (25° C.)

The polymer according to the invention is preferably not an elastomer.

The term “non-elastomeric polymer” is intended to mean a polymer which, when it is subjected to a stress intended for stretching it (for example by 30% relative to its initial length), does not return to a length substantially identical to its initial length when the stress stops.

More specifically, the term “non-elastomeric polymer” denotes a polymer which has an instantaneous recovery Rᵢ<50% and a delayed recovery R₂ₕ<70% after having undergone an elongation of 30%. Preferably, Rᵢ is <30%, and R₂ₕ<50%.

More specifically, the non-elastomeric nature of the polymer is determined according to the following protocol:

A film of polymer is prepared by pouring a solution of the polymer into a Teflon-coated template and then drying for 7 days in a controlled atmosphere at 23±5° C. and 50±10% relative humidity.

A film approximately 100 μm thick is then obtained, from which are cut rectangular testpieces (for example with a punch) that are 15 mm wide and 80 mm long.

A tensile stress is applied to this sample by means of a device sold under the reference Zwick, under the same temperature and humidity conditions as for the drying.

The testpieces are drawn at a speed of 50 mm/min and the distance between the jaws is 50 mm, which corresponds to the initial length (I₀) of the testpiece.

The instantaneous recovery Ri is determined in the following way:

the testpiece is drawn by 30% (εₘₐₓ), i.e. approximately 0.3 times its initial length (I₀);

the stress is released by applying a return speed equal to the tensile speed, i.e. 50 mm/min, and the percentage residual elongation of the testpiece is measured, after return to a zero constraint (εᵢ).

The % instantaneous recovery (Rᵢ) is given by the following formula:

Rᵢ=((εₘₐₓ−εᵢ)/εₘₐₓ)×100

To determine the delayed recovery, after 2 hours, the degree of residual elongation of the testpiece is measured as a percentage (ε₂ₕ), 2 hours after return to the zero constraint.

The % delayed recovery (R₂ₕ) is given by the following formula:

R₂ₕ=((εₘₐₓ−ε₂ₕ)/εₘₐₓ)×100

By way purely of illustration, a polymer according to one embodiment of the invention preferably has an instantaneous recovery Rᵢ of 10% and a delayed recovery R₂ₕ of 30%.

The polydispersity index of the polymer of the invention is greater than 2.

Advantageously, the block polymer used in the compositions according to the invention has a polydispersity index I greater than 2, for example ranging from 2 to 9, preferably greater than or equal to 2.5, for example ranging from 2.5 to 8, and better still greater than or equal to 2.8, and especially ranging from 2.8 to 6.

The polydispersity index I of the polymer is equal to the ratio of the weight-average mass Mw to the number-average mass Mn.

The weight-average (Mw) and number-average(Mn) molar masses are determined by gel permeation liquid chromatography (solvent THF, calibration curve established with standards of linear polystyrene, refractometric detector).

The weight-average mass (Mw) of the polymer according to the invention is preferably less than or equal to 300 000; it ranges, for example, from 35 000 to 200 000, and better still from 45 000 to 150 000 g/mol.

The number-average mass (Mn) of the polymer according to the invention is preferably less than or equal to 70 000; it ranges, for example, from 10 000 to 60 000, and better still from 12 000 to 50 000 g/mol.

Preferably, the polydispersity index of the polymer according to the invention is greater than 2, for example ranging from 2 to 9, preferably greater than or equal to 2.5, for example ranging from 2.5 to 8, and better still greater than or equal to 2.8, and especially ranging from 2.8 to 6.

First Block Having a Tg of Greater Than or Equal to 40° C.

The block having a Tg of greater than or equal to 40° C. has, for example, a Tg ranging from 40 to 150° C., preferably greater than or equal to 50° C., ranging, for example, from 50° C. to 120° C., and better still greater than or equal to 60° C., ranging, for example, from 60° C. to 120° C.

The indicated glass transition temperatures of the first and second blocks can be theoretical Tgs determined from the theoretical Tgs of the constitutive monomers of each of the blocks, which can be found in a reference manual such as the Polymer Handbook, 3rd Ed., 1989, John Wiley, according to the following relationship, known as Fox's law:

1/Tg=Σᵢ(ωᵢ/Tgᵢ),

ωᵢ being the fraction by mass of the monomer i in the block under consideration and Tgᵢ being the glass transition temperature of the homopolymer of the monomer i.

Unless otherwise indicated, the Tgs indicated for the first and second blocks in the present application are theoretical Tgs.

The difference between the glass transition temperatures of the first and second blocks is generally greater than 10° C., preferably greater than 20° C., and better still greater than 30° C.

In the present invention, the expression “between . . . and . . . ” is intended to mean a range of values of which the limits mentioned are excluded, and the expressions “from . . . to . . . ” and “ranging from . . . to . . . ” are intended to mean a range of values of which the limits are included.

The block having a Tg of greater than or equal to 40° C. can be a homopolymer or a copolymer.

The block having a Tg of greater than or equal to 40° C. can be completely or partially derived from one or more monomers, which are such that the homopolymer prepared from these monomers has a glass transition temperature of greater than or equal to 40° C. This block can also be referred to as a “rigid block”.

When this block is a homopolymer, it is derived from monomers, which are such that the homopolymers prepared from these monomers have glass transition temperatures of greater than or equal to 40° C. This first block may be a homopolymer, constituted of a single type of monomer (of which the Tg of the corresponding homopolymer is greater than or equal to 40° C.)

When the first block is a copolymer, it can be completely or partially derived from one or more monomers, the nature and the concentration of which are chosen such that the Tg of the resulting copolymer is greater than or equal to 40° C. The copolymer may, for example, comprise:

monomers which are such that the homopolymers prepared from these monomers have Tgs of greater than or equal to 40° C., for example a Tg ranging from 40° C. to 150° C., preferably greater than or equal to 50° C., ranging, for example, from 50° C. to 120° C., and better still greater than or equal to 60° C., ranging, for example, from 60° C. to 120° C., and

monomers which are such that the homopolymers prepared from these monomers have Tgs of less than 40° C., chosen from monomers having a Tg of between 20° C. and 40° C. and/or monomers having a Tg of less than or equal to 20° C., for example a Tg ranging from −100° C. to 20° C., preferably less than 15° C., in particular ranging from −80° C. to 15° C., and better still less than 10° C., for example ranging from −50° C. to 0° C., as described below.

The first monomers of which the homopolymers have a glass transition temperature of greater than or equal to 40° C. are preferably chosen from the following monomers, also called main monomers:

methacrylates of formula CH₂═C(CH₃)—COOR₁

in which R₁ represents a linear or branched, unsubstituted alkyl group containing from 1 to 4 carbon atoms, such as a methyl, ethyl, propyl or isobutyl group, or R₁ represents a C₄ to C₁₂ cycloalkyl group, preferably a C₈ to C₁₂ cycloalkyl, such as isobornyl methacrylate,

acrylates of formula CH₂═CH—COOR₂

in which R₂ represents a C₄ to C₁₂ cycloalkyl group, such as an isobornyl group or a tert-butyl group,

(meth)acrylamides of formula:

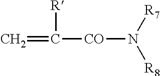

where R₇ and R₈, which may be identical or different, each represent a hydrogen atom or a linear or branched C₁ to C₁₂ alkyl group, such as an n-butyl, t-butyl, isopropyl, isohexyl, isooctyl or isononyl group; or R₇ represents H and R₈ represents a 1,1-dimethyl-3-oxobutyl group,

and R′ denotes H or methyl. As examples of monomers, mention may be made of N-butylacrylamide, N-t-butylacrylamide, N-isopropylacrylamide, N,N-dimethylacrylamide and N,N-dibutylacrylamide,

and mixtures thereof.

The first block is advantageously obtained from at least one acrylate monomer of formula CH₂═CH—COOR₂ and at least one methacrylate monomer of formula CH₂═C(CH₃)—COOR₂ in which R₂ represents a C₄ to C₁₂ cycloalkyl group, preferably a C₈ to C₁₂ cycloalkyl, such as isobornyl. The monomers and the proportions thereof are preferably chosen such that the glass transition temperature of the first block is greater than or equal to 40° C.

According to one embodiment, the first block is obtained from:
i) at least one acrylate monomer of formula CH₂═CH—COOR₂ in which R₂ represents a C₄ to C₁₂ cycloalkyl group, preferably a C₈ to C₁₂ cycloalkyl group, such as isobornyl,
ii) and at least one methacrylate monomer of formula CH₂═C(CH₃)—COOR′₂ in which R′₂ represents a C₄ to C₁₂ cycloalkyl group, preferably a C₈ to C₁₂ cycloalkyl group, such as isobornyl.

According to one embodiment, the first block is obtained from at least one acrylate monomer of formula CH₂═CH—COOR₂ in which R₂ represents a C₈ to C₁₂ cycloalkyl group, such as isobornyl, and at least one methacrylate monomer of formula CH₂═C(CH₃)—COOR′₂ in which R′₂ represents a C₈ to C₁₂ cycloalkyl group, such as isobornyl.

Preferably, R₂ and R′₂ independently or simultaneously represent an isobornyl group.

Preferably, the block copolymer comprises from 50% to 80% by weight of isobornyl methacrylate/acrylate, from 10% to 30% by weight of isobutyl acrylate and from 2% to 10% by weight of acrylic acid.

The first block may be obtained exclusively from said acrylate monomer and from said methacrylate monomer.

The acrylate monomer and the methacrylate monomer are preferably in proportions by mass of between 30:70 and 70:30, preferably between 40:60 and 60:40, in particular of the order of 50:50.

The proportion of the first block advantageously ranges from 20% to 90% by weight of the polymer, better still from 30% to 80%, and even better still from 60% to 80%.

According to one embodiment, the first block is obtained by polymerization of isobornyl methacrylate and isobornyl acrylate.

Second Block Having a Glass Transition Temperature of Less Than 20° C.

The second block advantageously has a glass transition temperature Tg of less than or equal to 20° C., for example has a Tg ranging from −100° C. to 20° C., preferably less than or equal to 15° C., in particular ranging from −80° C. to 15° C., and better still less than or equal to 10° C., for example ranging from −100° C. to 10° C., in particular ranging from −30° C. to 10° C.

The second block is completely or partially derived from one or more second monomers, which are such that the homopolymer prepared from these monomers has a glass transition temperature of less than or equal to 20° C.

This block can also be referred to as a “flexible block”.

The monomer having a Tg of less than or equal to 20° C. (referred to as second monomer) is preferably chosen from the following monomers:
acrylates of formula CH₂═CHCOOR₃,
R₃ representing a linear or branched, unsubstituted C₁ to C₁₂ alkyl group, with the exception of the tert-butyl group, in which one or more heteroatoms chosen from O, N and S is (are) optionally intercalated,
methacrylates of formula CH₂═C(CH₃)—COOR₄,
R₄ representing a linear or branched, unsubstituted C₆ to C₁₂ alkyl group, in which one or more heteroatoms chosen from O, N and S is (are) optionally intercalated,
vinyl esters of formula R₅—CO—O—CH═CH₂
where R₅ represents a linear or branched C₄ to C₁₂ alkyl group,
ethers of vinyl alcohol and of a C₄ to C₁₂ alcohol,
N—(C₄ to C₁₂)alkylacrylamides, such as N-octylacrylamide,
and mixtures thereof.

The monomers having a Tg of less than or equal to 20° C. which are preferred are isobutyl acrylate, 2-ethylhexyl acrylate or mixtures thereof in any proportions.

Each of the first and second blocks can contain, in a minor proportion, at least one constitutive monomer of the other block.

Thus, the first block can contain at least one constitutive monomer of the second block, and vice versa.

According to one preferred embodiment of the invention, said first monomer(s), which is (are) such that the homopolymer prepared from this or these monomer(s) has a glass transition temperature of greater than or equal to 40° C., is (are) chosen from:
methacrylates of formula CH₂═C(CH₃)—COOR₁
in which R₁ represents a linear or branched, unsubstituted alkyl group containing from 1 to 4 carbon atoms, or a C₄ to C₁₂ cycloalkyl group,
acrylates of formula CH₂═CH—COOR₂
in which R₂ represents a C₄ to C₁₂ cycloalkyl group,
(meth)acrylamides of formula:

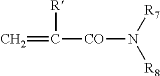

where R₇ and R₈, which may be identical or different, each represent a hydrogen atom or a linear or branched C₁ to C₁₂ alkyl group, or R₇ represents H and R₈ represents a 1,1-dimethyl-3-oxobutyl group,
and R′ denotes H or methyl,
   and said second monomer(s), which is (are) such that the homopolymer prepared from this or these monomer(s) has a glass transition temperature of less than or equal to 20° C., is (are) chosen from:
acrylates of formula CH₂═CHCOOR₃,
R₃ representing a linear or branched, unsubstituted C₁ to C₁₂ alkyl group, with the exception of the tert-butyl group, in which one or more heteroatoms chosen from O, N and S is (are) optionally intercalated,
methacrylates of formula CH₂═C(CH₃)—COOR₄,
R₄ representing a linear or branched, unsubstituted C₆ to C₁₂ alkyl group, in which one or more heteroatoms chosen from O, N and S is (are) optionally intercalated,
vinyl esters of formula R₅—CO—O—CH═CH₂
where R₅ represents a linear or branched C₄ to C₁₂ alkyl group,
ethers of vinyl alcohol and of a C₄ to C₁₂ alcohol,
N—(C₄ to C₁₂)alkylacrylamides, such as N-octylacrylamide,
and mixtures thereof.

Each of the first and/or second block(s) comprise(s), in addition to the monomers indicated above, one or more other monomers, called additional monomers, that are different from the main monomers mentioned above. In particular, each of the first and/or second block(s) comprise(s) at least one additional monomer. The nature and the amount of this or these additional monomer(s) are chosen in such a way that the block in which they are present has the desired glass transition temperature.

This additional monomer is, for example, chosen from:
monomers with one or more ethylenic unsaturation(s), comprising at least one tertiary amine function, such as 2-vinylpyridine, 4-vinylpyridine, dimethylaminoethyl methacrylate, diethylaminoethyl methacrylate, dimethylamino-propylmethacrylamide, and salts thereof,
methacrylates of formula CH₂═C(CH₃)—COOR₆
in which R₆ represents a linear or branched alkyl group containing from 1 to 4 carbon atoms, such as a methyl, ethyl, propyl or isobutyl group, said alkyl group being substituted with one or more substituents chosen from hydroxyl groups (such as 2-hydroxypropyl methacrylate or 2-hydroxyethyl methacrylate) and halogen atoms (Cl, Br, I, F), such as trifluoroethyl methacrylate,
methacrylates of formula CH₂═C(CH₃)—COOR₉,
R₉ representing a linear or branched C₆ to C₁₂ alkyl group in which one or more heteroatoms chosen from O, N and S is (are) optionally intercalated, said alkyl group being substituted with one or more substituents chosen from hydroxyl groups and halogen atoms (Cl, Br, I, F),
acrylates of formula CH₂═CHCOOR₁₀,
R₁₀ representing a linear or branched C₁ to C₁₂ alkyl group substituted with one or more substituents chosen from hydroxyl groups and halogen atoms (Cl, Br, I and F), such as 2-hydroxypropyl acrylate and 2-hydroxyethyl acrylate, or R₁₀ represents a (C₁ to C₁₂) alkyl-O-POE (polyoxyethylene) with repetition of the oxyethylene unit from 5 to 10 times, for example methoxy-POE, or R₈ represents a polyoxyethylenated group comprising from 5 to 10 ethylene oxide units.

In particular, the first block and/or the second block comprise(s), by way of additional monomer, (meth)acrylic acid, preferably acrylic acid.

The additional monomer(s) can represent from 1% to 60% by weight, relative to the total weight of the polymer, preferably from 3% to 30% by weight, relative to the total weight of said polymer.

In particular, said block copolymer is such that:

the first block is obtained from at least one acrylate monomer of formula CH₂═CH—COOR₂ in which R₂ represents a C₄ to C₁₂ cycloalkyl group, and at least one methacrylate monomer of formula CH₂═C(CH₃)—COOR′₂ in which R′₂ represents a C₄ to C₁₂ cycloalkyl group, and

the second block is obtained from at least one second monomer, such that the homopolymer obtained has a glass transition temperature of less than or equal to 20° C., and an additional monomer of acrylic acid type.

Preferably, the polymer of the invention comprises at least isobornyl acrylate and isobornyl methacrylate monomers in the first block and isobutyl acrylate and acrylic acid monomers in the second block.

Preferably, the polymer comprises at least isobornyl acrylate and isobornyl methacrylate monomers in equivalent proportion by weight in the first block and isobutyl acrylate and acrylic acid monomers in the second block.

Preferably, the polymer comprises at least isobornyl acrylate and isobornyl methacrylate monomers in equivalent proportion by weight in the first block, and isobutyl acrylate and acrylic acid monomers in the second block, the first block representing 70% by weight of the polymer.

Preferably, the polymer comprises at least isobornyl acrylate and isobornyl methacrylate monomers in equivalent proportion by weight in the first block, and isobutyl acrylate and acrylic acid monomers in the second block. Preferably, the block having a Tg of greater than 40° C. represents 70% by weight of the polymer, and the acrylic acid represents 5% by weight of the polymer.

According to one embodiment, the first block does not comprise an additional monomer.

According to one preferred embodiment, the second block comprises acrylic acid as additional monomer. In particular, the second block is advantageously obtained from an acrylic acid monomer and at least one other monomer having a Tg of less than or equal to 20° C.

According to one preferred embodiment, the invention relates to a cosmetic composition for making up and/or caring for keratin materials, comprising, in a physiologically acceptable medium, at least one copolymer comprising at least one acrylate monomer of formula CH₂═CH—COOR₂ in which R₂ represents a C₈ to C₁₂ cycloalkyl group and/or at least one methacrylate monomer of formula CH₂═C(CH₃)—COOR′₂ in which R′₂ represents a C₈ to C₁₂ cycloalkyl group, at least one second acrylate monomer of formula CH₂═CHCOOR₃ in which R₃ represents a linear or branched, unsubstituted C₁ to C₁₂ alkyl group, with the exception of the tert-butyl group, and at least one acrylic acid monomer.

Preferably, the copolymer used in the compositions according to the invention is obtained from at least one isobornyl methacrylate monomer, at least one isobornyl acrylate monomer, at least one isobutyl acrylate monomer and at least one acrylic acid monomer.

Advantageously, the copolymer used in the invention comprises from 50% to 80% by weight of isobornyl methacrylate/acrylate mixture, from 10% to 30% by weight of isobutyl acrylate and from 2% to 10% by weight of acrylic acid.

The block copolymer can advantageously comprise more than 2% by weight of acrylic acid monomers, and especially from 2% to 15% by weight, for example from 3% to 15% by weight, in particular from 4% to 15% by weight, or even from 4% to 10% by weight of acrylic acid monomers, relative to the total weight of said copolymer.

Intermediate Segment

The intermediate segment (also referred to as intermediate block) links the first block and the second block of the polymer used according to the present invention. The intermediate segment results from the polymerization:
i) of the first monomer(s), and optionally of the additional monomer(s), remaining available after their polymerization at a degree of conversion of at most 90% so as to form the first block,
ii) and of the second monomer(s) and optionally the additional monomer(s), added to the reaction mixture.

The formation of the second block is initiated when the first monomers no longer react or are no longer incorporated into the polymer chain, either because they are all consumed or because their reactivity no longer allows them to be incorporated.

Thus, the intermediate segment comprises the first monomers that are available, as a result of a degree of conversion of these first monomers of less than or equal to 90%, at the time of the introduction of the second monomer(s) during the synthesis of the polymer.

The intermediate segment of the block polymer is a random polymer (can also be referred to as a random block), i.e. it comprises a random distribution of the first monomer(s) and of the second monomer(s) and also of the additional monomer(s) optionally present.

Thus, the intermediate segment is a random block, as are the first block and the second block if they are not homopolymers (i.e. if they are both formed from at least two different monomers).

Process for Preparing the Copolymer:

The block ethylenic copolymer according to the invention can be prepared by free-radical polymerization, according to the well-known techniques of this type of polymerization.

The free-radical polymerization is carried out in the presence of an initiator, the nature of which is adjusted, in a known manner, according to the desired polymerization temperature and the polymerization solvent. In particular, the initiator can be chosen from initiators with a peroxide function, oxidation/reduction couples or other radical polymerization initiators known to those skilled in the art.

In particular, by way of initiator comprising a peroxide function, mention may, for example, be made of:
a. peroxyesters, such as tert-butyl peroxyacetate, tert-butyl perbenzoate, tert-butyl peroxy(2-ethylhexanoate) (Trigonox 21S from Akzo Nobel), 2,5-bis(2-ethylhexanoylperoxy)-2,5-dimethylhexane (Trigonox 141 from Akzo Nobel);
b. peroxydicarbonates, such as diisopropyl peroxy-dicarbonate;
c. peroxyketones, such as methyl ethyl ketone peroxide;
d. hydroperoxides, such as aqueous hydrogen peroxide solution (H₂O₂) or tert-butyl hydroperoxide;
e. diacyl peroxides, such as acetyl peroxide or benzoyl peroxide;
f. dialkyl peroxides, such as di(tert-butyl)peroxide;
g. inorganic peroxides, such as potassium peroxydisulphate (K₂S₂O₈).

By way of initiator in the form of an oxidation/reduction couple, mention may be made of the potassium thiosulphate+potassium peroxydisulphate couple, for example.

According to one preferred embodiment, the initiator is chosen from organic peroxides containing from 8 to 30 carbon atoms. Preferably, the initiator used is 2,5-bis(2-ethylhexanoylperoxy)-2,5-dimethyl-hexane sold under the reference Trigonox® 141 by the company Akzo Nobel.

The block copolymer used according to the invention is prepared by free-radical polymerization and not by controlled or living polymerization. In particular, the polymerization of the block ethylenic copolymer is carried out in the absence of control agents, and in particular in the absence of control agents conventionally used in living or controlled polymerization processes, such as nitroxides, alkoxyamines, dithioesters, dithiocarbamates, dithio-carbonates or xanthates, trithiocarbonates or copper-based catalysts, for example.

As indicated above, the intermediate segment is a random block, just like the first block and the second block if they are not homopolymers (i.e. if they are both formed from at least two different monomers).

The block copolymer can be prepared by free-radical polymerization, and in particular by means of a process which consists in mixing, in the same reactor, a polymerization solvent, an initiator, at least one monomer with a glass transition temperature of greater than or equal to 40° C., and at least one monomer with a glass transition temperature of less than or equal to 20° C., according to the following sequence:

a portion of the polymerization solvent and optionally a portion of the initiator and monomers of the first fluid addition are run into the reactor, which mixture is heated to a reaction temperature of between 60 and 120° C.,

said at least one first monomer with a Tg of greater than or equal to 40° C. and optionally a portion of the initiator are subsequently run in, in a first fluid addition, and are left to react for a time T corresponding to a degree of conversion of said monomers of at most 90%,

again polymerization initiator and said at least one second monomer with a glass transition temperature of less than or equal to 20° C. are subsequently run into the reactor, in a second fluid addition, and are left to react for a time T′, at the end of which the degree of conversion of said monomers reaches a plateau,

the reaction mixture is brought back to ambient temperature.

Preferably, the copolymer can be prepared by free-radical polymerization, in particular by means of a process which consists in mixing, in the same reactor, a polymerization solvent, an initiator, an acrylic acid monomer, at least one monomer with a glass transition temperature of less than or equal to 20° C., at least one acrylate monomer of formula CH₂═CH—COOR₂ in which R₂ represents a C₄ to C₁₂ cycloalkyl group, and at least one methacrylate monomer of formula CH₂═C(CH₃)—COOR′₂ in which R′₂ represents a C₄ to C₁₂ cycloalkyl group, according to the following sequence of steps:

a portion of the polymerization solvent and optionally a portion of the initiator and monomers of the first fluid addition are run into the reactor, which mixture is heated to a reaction temperature of between 60° C. and 120° C.,

said at least one acrylate monomer of formula CH₂═CH—COOR₂ and said at least one methacrylate monomer of formula CH₂═C(CH₃)—COOR′₂, as monomers with a Tg of greater than or equal to 40° C., and optionally a portion of the initiator, are subsequently run in, in a first fluid addition, and are left to react for a time T corresponding to a degree of conversion of said monomers of at most 90%,

again polymerization initiator, the acrylic acid monomer and said at least one monomer with a glass transition of less than or equal to 20° C. are subsequently run into the reactor, in a second fluid addition, and are left to react for a time T′, at the end of which the degree of conversion of said monomers reaches a plateau,

the reaction mixture is brought back to ambient temperature.

The term “polymerization solvent” is intended to mean a solvent or a mixture of solvents. In particular, mention may be made, by way of polymerization solvent which can be used, of:
ketones which are liquid at ambient temperature, such as methyl ethyl ketone, methyl isobutyl ketone, diisobutyl ketone, isophorone, cyclohexanone or acetone;
propylene glycol ethers which are liquid at ambient temperature, such as propylene glycol monomethyl ether, propylene glycol monomethyl ether acetate or dipropylene glycol mono(n-butyl)ether;
short-chain esters (having a total of 3 to 8 carbon atoms), such as ethyl acetate, methyl acetate, propyl acetate, n-butyl acetate or isopentyl acetate;
ethers which are liquid at ambient temperature, such as diethyl ether, dimethyl ether or dichlorodiethyl ether;
alkanes which are liquid at ambient temperature, such as decane, heptane, dodecane, isododecane, cyclohexane or isohexadecane;
cyclic aromatic compounds which are liquid at ambient temperature, such as toluene and xylene; aldehydes which are liquid at ambient temperature, such as benzaldehyde or acetaldehyde, and mixtures thereof.

The polymerization solvent can be chosen in particular from ethyl acetate, butyl acetate, alcohols such as isopropanol or ethanol, aliphatic alkanes, such as isododecane, and mixtures thereof. Preferably, the polymerization solvent is a mixture of butyl acetate and isopropanol or isododecane.

According to another embodiment, the copolymer can be prepared by free-radical polymerization according to a preparation process which consists in mixing, in the same reactor, a polymerization solvent, an initiator, at least one monomer with a glass transition of less than or equal to 20° C., and at least one monomer with a Tg of greater than or equal to 40° C., according to the following sequence of steps:

a portion of the polymerization solvent and optionally a portion of the initiator and monomers of the first fluid addition are run into the reactor, which mixture is heated to a reaction temperature of between 60 and 120° C.,

said at least one monomer with a glass transition of less than or equal to 20° C. and optionally a portion of the initiator are subsequently run in, in a first fluid addition, and are left to react for a time T corresponding to a degree of conversion of said monomers of at most 90%,

again polymerization initiator and said at least one monomer with a Tg of greater than or equal to 40° C. are subsequently run into the reactor, in a second fluid addition, and are left to react for a time T′, at the end of which the degree of conversion of said monomers reaches a plateau,

the reaction mixture is brought back to ambient temperature.

According to one preferred embodiment, the copolymer can be prepared by free-radical polymerization according to a preparation process which consists in mixing, in the same reactor, a polymerization solvent, an initiator, an acrylic acid monomer, at least one monomer with a glass transition of less than or equal to 20° C., at least one monomer with a Tg of greater than or equal to 40° C., and, in particular as monomers with a Tg of greater than or equal to 40° C., at least one acrylate monomer of formula CH₂═CH—COOR₂ in which R₂ represents a C₄ to C₁₂ cycloalkyl group and at least one methacrylate monomer of formula CH₂═C(CH₃)—COOR′₂ in which R′₂ represents a C₄ to C₁₂ cycloalkyl group, according to the following sequence of steps:

a portion of the polymerization solvent and optionally a portion of the initiator and monomers of the first fluid addition are run into the reactor, which mixture is heated to a reaction temperature of between 60 and 120° C.,

the acrylic acid monomer and said at least one monomer with a glass transition of less than or equal to 20° C. and optionally a portion of the initiator are subsequently run in, in a first fluid addition, and are left to react for a time T corresponding to a degree of conversion of said monomers of at most 90%,

again the polymerization initiator, said at least one acrylate monomer of formula CH₂═CH—COOR₂ and said at least one methacrylate monomer of formula CH₂═C(CH₃)—COOR′₂, as monomers with a Tg of greater than or equal to 40° C., are subsequently run into the reactor, in a second fluid addition, and are left to react for a time T′, at the end of which the degree of conversion of said monomers reaches a plateau,

the reaction mixture is brought back to ambient temperature.

The polymerization temperature is preferably of the order of 90° C.

The reaction time after the second fluid addition is preferably between 3 and 6 hours.

According to one particular embodiment of the invention, a poly(isobornyl acrylate/isobornyl methacrylate/isobutyl acrylate/acrylic acid) copolymer as prepared according to Example 1 described hereinafter will be used.

According to the invention, the block copolymer can be present in a content with respect to active material ranging from 0.1% to 30%, preferably between 0.5% and 20%, and even more preferably between 1% and 10% by weight of active material of block copolymer, relative to the total weight of said composition.

Fatty Phase

A cosmetic composition in accordance with the invention comprises at least one liquid fatty phase comprising at least one non-volatile oil as defined hereinafter, and a volatile oil.

The term “oil” is intended to mean any fatty substance which is in liquid form at ambient temperature (20-25° C.) and at atmospheric pressure.

The specific non-volatile oil according to the invention is a hydrocarbon-based oil and the volatile oil according to the invention can be chosen from hydrocarbon-based oils, silicone oils and fluoro oils, or mixtures thereof.

They may be of animal, plant, mineral or synthetic origin.

For the purpose of the present invention, the term “volatile oil” is intended to mean an oil (or nonaqueous medium) capable of evaporating on contact with the skin in less than one hour, at ambient temperature and at atmospheric pressure. The volatile oil is a volatile cosmetic oil, which is liquid at ambient temperature, and which has especially a non-zero vapour pressure, at ambient temperature and at atmospheric pressure, in particular which has a vapour pressure ranging from 0.13 Pa to 40 000 Pa (10⁻³ to 300 mmHg), and preferably ranging from 1.3 Pa to 13 000 Pa (0.01 to 100 mmHg), and preferentially ranging from 1.3 Pa to 1300 Pa (0.01 to 10 mmHg).

For the purpose of the present invention, the term “non-volatile oil” is intended to mean an oil which has a vapour pressure of less than 0.13 Pa.

For the purpose of the present invention, the term “silicone oil” is intended to mean an oil comprising at least one silicon atom, and in particular at least one Si—O group.

The term “fluoro oil” is intended to mean an oil comprising at least one fluorine atom.

The term “hydrocarbon-based oil” is intended to mean an oil containing mainly hydrogen and carbon atoms.

The oils can optionally comprise oxygen, nitrogen, sulphur and/or phosphorus atoms, for example in the form of hydroxyl or acid radicals or in the form of an ester function.

A composition of the invention can comprise a liquid fatty phase in a content ranging from 3% to 95%, in particular from 5% to 80%, in particular from 10% to 70%, and more particularly from 20% to 50% by weight, relative to the total weight of the composition.

Non-Volatile Oil

The composition of the invention comprises at least one hydrocarbon-based non-volatile oil characterized by a Hansen solubility parameter δa ranging from 2 to 7 (J/cm³)^{1/2} and a molecular mass of less than or equal to 300 g/mol.

Oils for which the Hansen solubility parameter δa is between 4 and 6 (J/cm³)^{1/2} and for which the molecular mass is less than 250 g/mol are preferred.

The definition and calculation of the solubility parameters in the three-dimensional Hansen solubility space are described in the article by C. M. Hansen: “The three dimensional solubility parameters” J. Paint Technol. 39, 105 (1967).

According to this Hansen space:

δ_{D} characterizes the London dispersion forces derived from the formation of dipoles induced during molecular impacts;

δₚ characterizes the Debye interaction forces between permanent dipoles and the Keesom interaction forces between induced dipoles and permanent dipoles;

δₕ characterizes the specific interaction forces (such as hydrogen bonding, acid/base, donor/acceptor, etc.);

δₐ is determined by the equation:

δₐ=(δₚ²+δₕ²)^{1/2}

The δₚ, δₕ, δ_{D} and δₐ parameters are expressed in (J/cm³)^{1/2}.

As examples of hydrocarbon-based non-volatile oils that may be suitable in the context of this invention, mention may be made of:

isodecyl neopentanoate (δa: 5.32; M: 242.4), isopropyl myristate (δa: 5.00; M: 270.46), isopropyl palmitate (δa: 4.74; M: 298.51), 2-ethylhexyl 2-ethylhexanoate (δa: 5.15; M: 256.43), isononyl isononanoate (δa: 4.57; M: 284.48), isodecyl isononanoate (δa: 4.74; M: 298.51), dicapryl carbonate (δa: 6.0; M: 286) and dicapryl ether (δa: 3.49; M: 242).

According to the invention, isodecyl neopentanoate is preferred.

The percentage of non-volatile oil is between 0.1% and 30%, preferably between 1% and 20%, and even more preferably between 2% and 10% by weight, relative to the total weight of said composition.

Preferably, the weight ratio of the copolymer to the non-volatile oil is between 0.5 and 100, and even more preferably between 1 and 40.

Volatile Oil

The composition of the invention also comprises at least one volatile oil which may be a hydrocarbon-based, silicone or fluoro oil.

The volatile oils can be chosen from hydrocarbon-based oils containing from 8 to 16 carbon atoms, and in particular branched C₈-C₁₆ alkanes (also known as isoparaffins), for instance isododecane (also known as 2,2,4,4,6-pentamethylheptane), isodecane, isohexadecane and, for example, the oils sold under the trade names Isopars® or Permethyls®. Mention may also be made of volatile linear alkanes containing from 9 to 15 carbon atoms, such as those described in Patent Application WO 2007/068371 A1 from the company Cognis.

As volatile oils, use may also be made of volatile silicones, for instance linear or cyclic volatile silicone oils, in particular those having a viscosity ≦8 centistokes (cSt) (8×10⁻⁶ m²/s), and having in particular from 2 to 10 silicon atoms, and in particular from 2 to 7 silicon atoms, these silicones optionally comprising alkyl or alkoxy groups containing from 1 to 10 carbon atoms. As a volatile silicone oil that can be used in the invention, mention may in particular be made of dimethicones of a viscosity of 5 and 6 cSt, octamethylcyclotetrasiloxane, decamethyl-cyclopentasiloxane, dodecamethylcyclohexasiloxane, heptamethylhexyltrisiloxane, octamethyltrisiloxane, decamethyltetrasiloxane and dodecamethylpentasiloxane, and mixtures thereof.

Use may also be made of volatile fluoro oils, such as nonafluoromethoxybutane, decafluoropentane, tetradecafluorohexane or dodecafluoropentane, and mixtures thereof.

Preferably, the volatile oil is a hydrocarbon-based volatile oil.

In particular, the volatile oils can be chosen from hydrocarbon-based oils containing from 8 to 14 carbon atoms, and in particular branched C₈-C₁₄ alkanes, for instance isoalkanes of petroleum origin, such as isododecane and isodecane.

The percentage of volatile oil is between 1% and 80%, preferably between 10% and 60%, and even more preferably between 20% and 50% by weight, relative to the total weight of said composition.

Physiologically Acceptable Medium

The term “physiologically acceptable medium” is intended to denote a medium which is particularly suitable for the application of a composition of the invention to the skin or the lips.

The physiologically acceptable medium is generally adjusted to the nature of the substrate on which the composition must be applied, and also to the way in which the composition must be packaged.

A composition of the invention may be anhydrous or in the form of a dispersion or an emulsion.

An emulsion can have an oily or aqueous continuous phase. Such an emulsion may, for example, be an inverse (W/O) or direct (O/W) emulsion or else a multiple emulsion (W/O/W or O/W/O).

In the case of emulsions, inverse emulsions (W/O) are preferred.

Lipophilic Structuring Agent

A composition according to the invention may also comprise at least one agent for structuring a liquid fatty phase, selected from a wax, a pasty compound, and mixtures thereof.

In particular, a wax suitable for the invention may especially be selected from waxes of animal, plant, mineral and synthetic origin, and mixtures thereof.

By way of examples of waxes that can be used according to the invention, mention may be made of:

waxes of animal origin, such as beeswax, spermaceti, lanolin wax and lanolin derivatives, plant waxes such as carnauba wax, candelilla wax, ouricury wax, Japan wax, cocoa butter, cork fibre wax or sugarcane wax,

mineral waxes, for example paraffin wax, petroleum jelly wax, lignite wax or microcrystalline waxes or ozokerites,

synthetic waxes, among which are polyethylene waxes, and waxes obtained by Fisher-Tropsch synthesis,

silicone waxes, in particular substituted linear polysiloxanes; mention may, for example, be made of silicone polyether waxes, alkyl dimethicones or alkoxy dimethicones having from 16 to 45 carbon atoms, alkyl methicones such as the C₃₀-C₄₅ alkyl methicone sold under the trade name “AMS C 30” by Dow Corning,

hydrogenated oils that are solid at 25° C., such as hydrogenated castor oil, hydrogenated jojoba oil, hydrogenated palm oil, hydrogenated tallow or hydrogenated coconut oil, and fatty esters that are solid at 25° C., for instance the C₂₀-C₄₀ alkyl stearate sold under the trade name “Kester Wax K82H” by the company Koster Keunen,

and/or mixtures thereof.

Preferably, polyethylene waxes, microcrystalline waxes, carnauba waxes, hydrogenated jojoba oil, candelilla waxes, beeswaxes and/or mixtures thereof will be used.

A composition according to the invention may also comprise at least one pasty compound.

The presence of a pasty compound may make it possible to advantageously confer improved comfort when a composition of the invention is deposited on keratin fibres.

Such a compound may be advantageously selected from lanolin and derivatives thereof; polymeric or nonpolymeric silicone compounds; polymeric or nonpolymeric fluoro compounds; vinyl polymers, in particular olefin homopolymers; olefin copolymers; hydrogenated diene homopolymers and copolymers; linear or branched and homo- or copolymeric oligomers of alkyl(meth)acrylates preferably having a C₈-C₃₀ alkyl group; homo- and copolymeric oligomers of vinyl esters having C₈-C₃₀ alkyl groups; homo- and copolymeric oligomers of vinyl ethers having C₈-C₃₀ alkyl groups; liposoluble polyethers resulting from polyetherification between one or more C₂-C₁₀₀, in particular C₂-C₅₀ diols; fatty acid or alcohol esters; and mixtures thereof.

Among the esters, mention may in particular be made of:

the esters of an oligomeric glycerol, especially the esters of diglycerol, for instance polyglyceryl-2 triisostearate, the condensates of adipic acid and of glycerol, for which a portion of the hydroxyl groups of the glycerols have reacted with a mixture of fatty acids, such as stearic acid, capric acid, stearic acid and isostearic acid and 12-hydroxystearic acid, such as in particular those sold under the trade mark Softisan 649 by the company Sasol, or such as bisdiglyceryl polyacyladipate-2; the arachidyl propionate sold under the trade mark Waxenol 801 by Alzo; phytosterol esters; triglycerides of fatty acids and derivatives thereof, such as hydrogenated cocoglycerides; noncrosslinked polyesters resulting from polycondensation between a linear or branched C₄-C₅₀ dicarboxylic acid or polycarboxylic acid and a C₂-C₅₀ diol or polyol; aliphatic esters of an ester resulting from the esterification of an aliphatic hydroxycarboxylic acid ester with an aliphatic carboxylic acid; polyesters resulting from the esterification, with a polycarboxylic acid, of an aliphatic hydroxycarboxylic acid ester, said ester comprising at least two hydroxyl groups, such as the products Risocast DA-H® and Risocast DA-L®; and mixtures thereof.

The structuring agent(s) may be present in a composition of the invention in a content ranging from 0.1% to 30% by weight of agents, more preferably from 0.5% to 20% by weight, relative to the total weight of the composition.

Aqueous Phase

The composition according to the invention may comprise an aqueous phase.

The aqueous phase comprises water. A water suitable for the invention may be a floral water such as cornflower water and/or a mineral water such as Vittel water, Lucas water or La Roche Posay water and/or a spring water.

The aqueous phase may also comprise organic solvents that are water-miscible (at ambient temperature—25° C.), for instance monoalcohols having from 2 to 6 carbon atoms, such as ethanol or isopropanol; polyols having in particular from 2 to 20 carbon atoms, preferably having from 2 to 10 carbon atoms, and preferentially having from 2 to 6 carbon atoms, such as glycerol, propylene glycol, butylene glycol, pentylene glycol, hexylene glycol, dipropylene glycol or diethylene glycol; glycol ethers (having in particular from 3 to 16 carbon atoms), such as mono-, di- or tripropylene glycol(C₁-C₄)alkyl ethers, mono-, di- or triethylene glycol(C₁-C₄)alkyl ethers, and mixtures thereof.

The aqueous phase may also comprise stabilizers, for example sodium chloride, magnesium dichloride and magnesium sulphate.

The aqueous phase may also comprise any water-soluble or water-dispersible compound compatible with an aqueous phase, such as gelling agents, film-forming polymers, thickeners, surfactants and mixtures thereof.

When the composition of the invention comprises an aqueous phase, it can be present in a content ranging from 1% to 80% by weight, especially from 5% to 50%, and more particularly from 10% to 45% by weight, relative to the total weight of the composition.

According to another embodiment, a composition of the invention may be anhydrous.

An anhydrous composition may comprise less than 5% by weight of water, relative to the total weight of the composition, and in particular less than 3%, especially less than 2%, and more particularly less than 1% by weight of water, relative to the total weight of the composition.

More particularly, an anhydrous composition may be free of water.

Thickeners

Depending on the fluidity of the composition that it is desired to obtain, one or more thickeners or gelling agents may be incorporated into a composition of the invention.

A thickener or gelling agent suitable for the invention may be hydrophilic, i.e. water-soluble or water-dispersible.

As hydrophilic gelling agents, mention may in particular be made of water-soluble or water-dispersible thickening polymers. Said polymers may in particular be selected from: modified or unmodified carboxyvinyl polymers, such as the products sold under the name Carbopol (CTFA name: carbomer) by the company Goodrich; polyacrylates and polymethacrylates, such as the products sold under the names Lubrajel and Norgel by the company Guardian or under the name Hispagel by the company Hispano Chimica; polyacrylamides; 2-acrylamido-2-methylpropanesulphonic acid polymers and copolymers, which are optionally crosslinked and/or neutralized, such as the poly(2-acrylamido-2-methylpropanesulphonic acid) sold by the company Clariant under the name “Hostacerin AMPS” (CTFA name: ammonium polyacryldimethyltauramide); crosslinked anionic acrylamide/AMPS copolymers, in the form of a W/O emulsion, such as those sold under the name Sepigel 305 (CTFA name: Polyacrylamide/C13-14 Isoparaffin/Laureth-7) and under the name Simulgel 600 (CTFA name: Acrylamide/Sodium acryloyldimethyltaurate copolymer/Isohexadecane/Polysorbate 80) by the company SEPPIC; polysaccharide biopolymers, such as xanthan gum, guar gum, carob gum, gum acacia, scleroglucans, chitin derivatives and chitosan derivatives, carrageenans, gellans, alginates, or celluloses such as microcrystalline cellulose, carboxymethylcellulose, hydroxymethylcellulose and hydroxypropylcellulose; and mixtures thereof.

A thickener or gelling agent suitable for the invention may be lipophilic. It may be mineral or organic.

As lipophilic thickeners, mention may, for example, be made of modified clays, such as modified magnesium silicate (Bentone gel VS38 from Rheox), modified hectorites such as hectorite modified with a C₁₀ to C₂₂ fatty acid ammonium chloride, for instance hectorite modified with distearyldimethylammonium chloride, for instance the product sold under the name Bentone 38V® by the company Elementis or the product sold under the name “Bentone 38 CE” by the company Rheox or the product sold under the name Bentone Gel V5 5V by the company Elementis.

The polymeric organic lipophilic gelling agents are, for example, partially or totally crosslinked elastomeric organopolysiloxanes with a three-dimensional structure, such as those sold under the names KSG6®, KSG16® and KSG18® by the company Shin-Etsu, Trefil E-505C® and Trefil E-506C® by the company Dow-Corning, Gransil SR-CYC®, SR DMF10®, SR-DC556®, SR 5CYC gel®, SR DMF 10 gel® and SR DC 556 gel® by the company Grant Industries and SF 1204® and JK 113® by the company General Electric; ethylcellulose, such as the product sold under the name Ethocel® by the company Dow Chemical; polyamide-type polycondensates resulting from condensation between a dicarboxylic acid containing at least 32 carbon atoms and an alkylene diamine, and in particular ethylene diamine, in which the polymer comprises at least one terminal carboxylic acid group esterified or amidified with at least one monoalcohol or one monoamine containing from 12 to 30 carbon atoms, and linear and saturated, and in particular ethylenediamine/stearyl dilinoleate copolymers such as the product sold under the name Uniclear 100 VG® by the company Arizona Chemical; galactomannans containing from one to six, and in particular from two to four, hydroxyl groups per monosaccharide, substituted with a saturated or unsaturated alkyl chain, such as guar gum alkylated with C₁ to C₆, and in particular C₁ to C₃, alkyl chains, and mixtures thereof. Block copolymers of “diblock”, “triblock” or “radial” type, of the polystyrene/polyisoprene or polystyrene/polybutadiene type, such as those sold under the name Luvitol HSB® by the company BASF, of the polystyrene/copoly(ethylene-propylene) type, such as those sold under the name Kraton® by the company Shell Chemical Co, or else of the polystyrene/copoly(ethylene-butylene) type, blends of triblock and radial (star) copolymers in isododecane, such as those sold by the company Penreco under the name Versagel®, for instance the mixture of butylene/ethylene/styrene triblock copolymer and of ethylene/propylene/styrene star copolymer in isododecane (Versagel M 5960).

Among the lipophilic gelling agents that can be used in a cosmetic composition of the invention, mention may also be made of esters of dextrin and of a fatty acid, such as dextrin palmitates, in particular such as those sold under the names Rheopearl TL® or Rheopearl KL® by the company Chiba Flour, hydrogenated plant oils, such as hydrogenated castor oil, fatty alcohols, in particular C₈ to C₂₆, and more particularly C₁₂ to C₂₂ fatty alcohols, for instance myristyl alcohol, cetyl alcohol, stearyl alcohol and behenyl alcohol.

According to one embodiment, a composition of the invention may comprise thickeners in a content with respect to active material of from 0.01% to 40% by weight, especially from 0.1% to 20% by weight, in particular from 1% to 15% by weight, relative to the total weight of the composition.

Colorants

According to one preferred embodiment, the composition according to the invention also comprises at least one colorant, in particular at least one pulverulent colorant.

A cosmetic composition in accordance with the invention may advantageously incorporate at least one colorant chosen from organic or inorganic colorants, in particular such as pigments or nacres conventionally used in cosmetic compositions, liposoluble or water-soluble dyes, materials with a specific optical effect, and mixtures thereof.

The term “pigments” should be understood to mean white or coloured, inorganic or organic particles which are insoluble in an aqueous solution and are intended for colouring and/or opacifying the resulting film.

The pigments may be present in a proportion of from 0.1% to 40% by weight, especially from 1% to 30% by weight, and in particular from 5% to 15% by weight, relative to the total weight of the cosmetic composition.

As inorganic pigments that can be used in the invention, mention may be made of titanium dioxide, zirconium oxide or cerium oxide, and also zinc oxide, iron oxide or chromium oxide, Prussian blue, manganese violet, ultramarine blue and chromium hydrate.

Among the organic pigments that can be used in the invention, mention may be made of carbon black, D & C pigments, lakes based on cochineal carmine, on barium, strontium, calcium or aluminium, or else diketopyrrolopyrroles (DPP) described in documents EP-A-542 669, EP-A-787 730, EP-A-787 731 and WO-A-96/08537.

The term “nacres” should be understood to mean iridescent or noniridescent coloured particles of any shape, which are in particular produced by certain molluscs in their shell or else are synthesized, and which exhibit a colour effect by optical interference.

The nacres may be chosen from pearlescent pigments, such as titanium mica coated with iron oxide, titanium mica coated with bismuth oxychloride, titanium mica coated with chromium oxide, titanium mica coated with an organic dye, and pearlescent pigments based on bismuth oxychloride. This may also involve mica particles at the surface of which are superposed at least two successive layers of metal oxides and/or of organic colorants.

By way of example of nacres, mention may also be made of natural mica coated with titanium oxide, with iron oxide, with natural pigment or with bismuth oxychloride.

The cosmetic composition according to the invention may also comprise water-soluble or liposoluble dyes. The liposoluble dyes are, for example, Sudan red, DC Red 17, DC Green 6, β-carotene, soya oil, Sudan brown, DC Yellow 11, DC Violet 2, DC orange 5 and quinoline yellow. The water-soluble dyes are, for example, beetroot juice and caramel.

The cosmetic composition according to the invention may also contain at least one material with a specific optical effect.

This effect is different from a simple, conventional hue effect, i.e. a unified and stabilized effect of the kind produced by conventional colorants, such as, for example, monochromatic pigments. For the purpose of the invention, the term “stabilized” signifies an absence of an effect of variability of colour with the angle of observation or else in response to a change in temperature.

For example, this material may be chosen from particles having a metallic glint, goniochromatic colouring agents, diffracting pigments, thermochromatic agents, optical brighteners, and also fibres, in particular of interference type. Of course, these various materials may be combined so as to provide the simultaneous manifestation of two effects, or even a new effect in accordance with the invention.

The metal-glint particles that can be used in the invention are in particular chosen from:

particles of at least one metal and/or of at least one metal derivative,

particles comprising a single-substance or multi-substance, organic or inorganic substrate, at least partially coated with at least one metal-glint layer comprising at least one metal and/or at least one metal derivative, and

mixtures of said particles.

Among the metals that may be present in said particles, mention may, for example, be made of Ag, Au, Cu, Al, Ni, Sn, Mg, Cr, Mo, Ti, Zr, Pt, Va, Rb, W, Zn, Ge, Te, Se and mixtures or alloys thereof. Ag, Au, Cu, Al, Zn, Ni, Mo, Cr and mixtures or alloys thereof (for example, bronzes and brasses) are preferred metals.

Fillers

A composition according to the invention may also comprise at least one filler, of organic or inorganic nature.

The term “filler” should be understood to mean colourless or white, solid particles of any shape, which are in a form that is insoluble or dispersed in the medium of the composition. Inorganic or organic in nature, they make it possible to confer, on the composition, softness, mattness and uniformity when making up.

The fillers used in the compositions according to the invention may be of lamellar, globular or spherical form, or in the form of fibres or in any other form intermediate between these defined forms.

The fillers according to the invention can optionally be surface-coated, and in particular they can be surface-treated with silicones, amino acids, fluoro derivatives or any other substance that promotes the dispersion and compatibility of the filler in the composition.

Among the inorganic fillers that can be used in the compositions according to the invention, mention may be made of talc, mica, silica, trimethyl siloxysilicate, kaolin, bentone, calcium carbonate and magnesium hydrogen carbonate, hydroxyapatite, boron nitride, hollow silica microspheres (silica beads from Maprecos), glass or ceramic microcapsules, silica-based fillers such as Aerosil 200 or Aerosil 300; Sunsphere H-33, Sunsphere H-51, sold by Asahi Glass; Chemicelen sold by Asahi Chemical; composites of silica and of titanium dioxide, such as the TSG series sold by Nippon Sheet Glass, and mixtures thereof.

Among the organic fillers that can be used in the compositions according to the invention, mention may be made of polyamide powders (Nylon® Orgasol from Atochem), poly-β-alanine and polyethylene powders, polytetrafluoroethylene (Teflon®) powders, lauroyllysine, starch, tetrafluoroethylene polymer powders, hollow microspheres of polymers, such as Expancel (Nobel Industrie), metal soaps derived from organic carboxylic acids having from 8 to 22 carbon atoms, preferably from 12 to 18 carbon atoms, for example zinc stearate, magnesium stearate or lithium stearate, zinc laurate, magnesium myristate, Polypore® L 200 (Chemdal Corporation), silicone resin microbeads (Tospearl® from Toshiba for example), polyurethane powders, in particular powders of crosslinked polyurethane comprising a copolymer, said copolymer comprising trimethylol hexyllactone, for instance the hexamethylene diisocyanate/trimethylol hexyllactone polymer sold under the name Plastic Powder D-400® or Plastic Powder D-800® by the company Toshiki, carnauba microwaxes, such as the product sold under the name MicroCare 350® by the company Micro Powders, microwaxes of synthetic wax, such as the product sold under the name MicroEase 114S® by the company Micro Powders, microwaxes constituted of a mixture of carnauba wax and polyethylene wax, such as those sold under the names Micro Care 300® and 310® by the company Micro Powders, microwaxes constituted of a mixture of carnauba wax and synthetic wax, such as the product sold under the name Micro Care 325® by the company Micro Powders, polyethylene microwaxes, such as those sold under the names Micropoly 200®, 220®, 220L® and 250S® by the company Micro Powders; and mixtures thereof.

The fillers may be present in a content ranging from 0.1% to 20% by weight, preferably from 0.5% to 15% by weight, relative to the total weight of said composition.

Additives

A cosmetic composition according to the invention may also further comprise any additive normally used in the field under consideration, for example chosen from gums, anionic, cationic, amphoteric or nonionic surfactants, silicone surfactants, resins, dispersants, semicrystalline polymers, antioxidants, essential oils, preservatives, fragrances, neutralizing agents, antiseptics, anti-UV protective agents, cosmetic active agents, such as vitamins, moisturizing agents, emollients, and mixtures thereof.

Those skilled in the art can adjust the nature and the amount of the additives present in the compositions in accordance with the invention by means of routine procedures, such that the cosmetic properties and the stability properties desired for these compositions are not thereby affected.

A composition according to the invention may in particular be in the form of a care or makeup composition, in particular a makeup composition for the skin, the lips or else the eyelashes.

According to one preferred embodiment, the composition according to the invention is a foundation.

The present invention is presented in greater detail in the examples described hereinafter, which are proposed only by way of illustration of the invention and should not be interpreted as limiting the invention. The values are expressed as % by weight.

### EXAMPLES

### Example 1

### Preparation of a poly(isobornyl acrylate/isobornyl methacrylate/isobutyl acrylate/acrylic acid) copolymer

300 g of isododecane are placed in a 1 litre reactor, and then the temperature is increased so as to go from ambient temperature (25° C.) to 90° C. over 1 hour.

105 g of isobornyl methacrylate, 105 g of isobornyl acrylate and 1.8 g of 2,5-bis(2-ethyl-hexanoylperoxy)-2,5-dimethylhexane (Trigonox® 141 from Akzo Nobel) are then added, at 90° C. and over 1 hour.

The mixture is kept at 90° C. for 1 h 30.

75 g of isobutyl acrylate, 15 g of acrylic acid and 1.2 g of 2,5-bis(2-ethylhexanoylperoxy)-2,5-dimethylhexane are then introduced into the previous mixture, still at 90° C. and over 30 minutes.

The mixture is kept at 90° C. for 3 hours and then the whole is cooled.

A solution containing 50% of active material of copolymer in isododecane is obtained.

A copolymer is obtained which comprises a first poly(isobornyl acrylate/isobornyl methacrylate) block having a Tg of 128° C., a second poly(isobutyl acrylate/acrylic acid) block having a Tg of −9° C. and an intermediate block which is an isobornyl acrylate/isobornyl methacrylate/isobutyl acrylate/acrylic acid random copolymer.

The Tg of the copolymer is 74° C.

These are theoretical Tgs calculated using Fox's law.

### Examples 2 to 5

### W/O Foundation: Influence of the Nature of the Non-Volatile Oil

| | | % by mass |
|---|---|---|
| A1 | PEG-30 Dipolyhydroxy stearate sold | 4.00 |
| | under the reference ARLACEL P 135 by | |
| | the company UNIQEMA | |
| | Non-volatile oil | 1.00 |
| | n-Undecane: n-tridecane mixture, the | 12.00 |
| | amount of n-undecane being in the | |
| | majority in the mixture* | |
| A2 | Bentone gel sold under the reference | 3.00 |
| | Bentone Gel ISD V by the company | |
| | Elementis | |
| A3 | Non-volatile oil | 3.00 |
| | Yellow iron oxide coated with aluminium | 1.79 |
| | stearoyl glutamate | |
| | Yellow iron oxide coated with aluminium | 0.54 |
| | stearoyl glutamate | |
| | Black iron oxide coated with aluminium | 0.19 |
| | stearoyl glutamate | |
| | Titanium dioxide coated with aluminium | 7.48 |
| | stearoyl glutamate | |
| A4 | Isododecane | 9.40 |
| | Poly(isobornyl methacrylate-co- | 16.00 |
| | isobornyl acrylate-co-isobutyl | |
| | acrylate-co-acrylic acid) containing | |
| | 50% of active material in isododecane | |
| A5 | Silica microsphere sold under the | 5.50 |
| | reference SB 700 by the company | |
| | Miyoshi Kasei | |
| A6 | Fragrance | 0.30 |
| B | Demineralized water | 27.50 |
| | Methyl paraben | 0.20 |
| | PEG 20 | 1.70 |
| | Magnesium sulphate | 0.70 |
| | Phenoxyethanol | 0.70 |
| C | Ethanol | 5.00 |
| | TOTAL | 100% by |
| | | mass |

*As prepared according to application WO2008/155059

| | | | | Example 5 |
|---|---|---|---|---|
| | Example 2 | Example 3 | Example 4 | (com- |
| | (invention) | (invention) | (comparative) | parative) |
| Nature of | Isodecyl | Dicapryl | Isohexa- | Octyl- |
| the non- | neopentanoate | ether sold | decane | dodecanol |
| volatile | | under the | | |
| oil | | reference | | |
| | | CETIOL OE | | |
| | | by the | | |
| | | company | | |
| | | COGNIS | | |
| δa (J/cm³)^{1/2} | 5.32 | 3.49 | 0 | 7.73 |
| M (g/mol) | 242.4 | 242 | 226.45 | 298.55 |

Procedure

The constituents of phase A1 are introduced into the main beaker, and are heated at 50-55° C. until a homogeneous liquid mixture is obtained.

Phase A2 is then introduced at ambient temperature and with stirring (Moritz stirrer: 1000 rpm), until a homogeneous mixture is obtained.

Phase A3 is prepared separately by milling the mixture of pigments and non-volatile oil three times on a triple roll mill. This phase A3 is added with stirring, and then phase A4, which was prepared separately by diluting the polymer with isododecane, and phase A5 and phase A6 are successively introduced.

The aqueous phase B is prepared in the following way: the methyl paraben, the magnesium sulphate and the PEG-20 are weighed out into a beaker. The water, brought to boiling beforehand and the temperature of which is in the region of 85-90° C., is then added, and then the mixture is stirred using a magnetic bar until the three constituents have dissolved. The temperature is allowed to drop back down to 40° C. and the phenoxyethanol is added.

The emulsion is prepared at ambient temperature: the aqueous phase B is poured into the fatty phase, with the stirring (Moritz) being gradually increased to 4500 rpm. This stirring is maintained for 10 min and, finally, phase C (ethanol) is added.

The product obtained is stirred with a Rayneri mixer (paddle mixer), stirring being carried out for 10 min at 100 rpm.

Measurement of the Mattness and of the Staying Power of the Mattness

Principle of the Measurement

The mattness of the face is measured using a polarimetric camera, which is a black and white polarimetric imaging system, with which images are acquired in parallel (P) and crossed (C) polarized light. By analysing the image resulting from the subtraction of the two images (P-C), the shine is quantified by measuring the mean level of grey of the shiniest 5% of pixels corresponding to the regions of shine.

Progression of the Test

The test progresses in the following way:

16 women arrive in an air-conditioned (22° C.+/−2° C.) waiting room 15 min before the beginning of the test.

They remove their makeup and an image of one of their cheeks is acquired with the polarimetric camera. This image makes it possible to measure the shine at T0 before applying makeup.

100 mg of foundation are then weighed out into a watch glass and are applied with the bare fingers to the half of the face on which the T0 measurement was carried out.

After a drying time of 15 min, an image of the made-up cheek is then acquired with the polarimetric camera. This image makes it possible to measure the shine immediately after applying makeup (Timm).

The models then return to the air-conditioned room for 3 h.

Finally, an image of the made-up cheek after waiting for 3 h is acquired with the polarimetric camera. This image makes it possible to measure the shine after wearing makeup for 3 h (T3h).

Expression of the Results

The difference (Timm−T0), which measures the effect of the makeup, is calculated. A negative value means that the makeup reduces the shine of the skin and that it is therefore mattifying.

The difference (T3h−Timm) which measures the staying power of this effect is subsequently calculated. The value obtained should be as low as possible, meaning that the mattness of the makeup does not change over time.

| | | | | Example 5 |
|---|---|---|---|---|
| | Example 2 | Example 3 | Example 4 | (com- |
| | (inventive) | (inventive) | (comparative) | parative) |
| Nature of | Isodecyl | Dicapryl | Isohexa- | Octyl- |
| the non- | neopentanoate | ether | decane | dodecanol |
| volatile oil | | sold | | |
| | | under the | | |
| | | reference | | |
| | | CETIOL OE | | |
| | | by the | | |
| | | company | | |
| | | COGNIS | | |
| Mattness | −3.85 | −3.36 | −5.97 | −5.74 |
| (Timm-T0) | | | | |
| Staying | 3.52 | 4.37 | 7.32 | 6.43 |
| power of the | | | | |
| mattness | | | | |
| (T3h-Timm) | | | | |

These results show that the non-volatile oils of the invention (Ex. 2 and 3) result in better staying power performance levels.

The above written description of the invention provides a manner and process of making and using it such that any person skilled in this art is enabled to make and use the same, this enablement being provided in particular for the subject matter of the appended claims, which make up a part of the original description.

As used herein, the phrases “selected from the group consisting of,” “chosen from,” and the like include mixtures of the specified materials. Terms such as “contain(s)” and the like as used herein are open terms meaning ‘including at least’ unless otherwise specifically noted. The term “mentioned” notes exemplary embodiments, and is not limiting to certain species. As used herein the words “a” and “an” and the like carry the meaning of “one or more.”

All references, patents, applications, tests, standards, documents, publications, brochures, texts, articles, etc. mentioned herein are incorporated herein by reference. Where a numerical limit or range is stated, the endpoints are included. Also, all values and subranges within a numerical limit or range are specifically included as if explicitly written out.

The above description is presented to enable a person skilled in the art to make and use the invention, and is provided in the context of a particular application and its requirements. Various modifications to the preferred embodiments will be readily apparent to those skilled in the art, and the generic principles defined herein may be applied to other embodiments and applications without departing from the spirit and scope of the invention. Thus, this invention is not intended to be limited to the embodiments shown, but is to be accorded the widest scope consistent with the principles and features disclosed herein. In this regard, certain embodiments within the invention may not show every benefit of the invention, considered broadly.

1. A composition comprising:
a) one block ethylenic copolymer comprising at least a first block having a glass transition temperature (Tg) of greater than or equal to 40° C. and being completely or partially derived from one or more first monomers, which are such that the homopolymer prepared from these monomers has a glass transition temperature of greater than or equal to 40° C., and at least one second block having a glass transition temperature of less than or equal to 20° C. and being completely or partially derived from one or more second monomers, which are such that the homopolymer prepared from these monomers has a glass transition temperature of less than or equal to 20° C., said first block and said second block being linked together via a random intermediate segment comprising at least one of said first constituent monomers of the first block and at least one of said second constituent monomers of the second block, and said block copolymer having a polydispersity index I of greater than 2,
b) a hydrocarbon-based non-volatile oil characterized by a Hansen solubility parameter δa ranging from 2 to 7 (J/cm³)^{1/2} and a molecular mass of less than or equal to 300 g/mol, and
c) a volatile oil.

2. The composition according to claim 1, wherein said composition is in the form of an emulsion.

3. The composition according to claim 1, wherein said first monomer(s), which is (are) such that the homopolymer prepared from this or these monomer(s) has a glass transition temperature of greater than or equal to 40° C., is (are) chosen from:
methacrylates of formula CH₂═C(CH₃)—COOR₁
in which R₁ represents a linear or branched, unsubstituted alkyl group containing from 1 to 4 carbon atoms, or a C₄ ^{to} C₁₂ cycloalkyl group,
acrylates of formula CH₂═CH—COOR₂
in which R₂ represents a C₄ to C₁₂ cycloalkyl group,
(meth)acrylamides of formula: 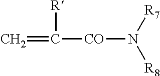
where R₇ and R₈, which may be identical or different, each represent a hydrogen atom or a linear or branched C₁ to C₁₂ alkyl group, or R₇ represents H and R₈ represents a 1,1-dimethyl-3-oxobutyl group,
and R′ denotes H or methyl,
and in that said second monomer(s), which is (are) such that the homopolymer prepared from this or these monomer(s) has a glass transition temperature of less than or equal to 20° C., is (are) chosen from:
acrylates of formula CH₂═CHCOOR₃,
R₃ representing a linear or branched, unsubstituted C₁ to C₁₂ alkyl group, with the exception of the tert-butyl group, in which one or more heteroatoms chosen from O, N and S is (are) optionally intercalated,
methacrylates of formula CH₂═C(CH₃)—COOR₄,
R₄ representing a linear or branched, unsubstituted C₆ to C₁₂ alkyl group in which one or more heteroatoms chosen from O, N and S is (are) optionally intercalated,
vinyl esters of formula R₅—CO—O—CH═CH₂
where R₅ represents a linear or branched C₄ to C₁₂ alkyl group,
ethers of vinyl alcohol and of a C₄ to C₁₂ alcohol,
N—(C₄ to C₁₂)alkylacrylamides, such as N-octyl-acrylamide,
and mixtures thereof.

4. The composition according to claim 3, wherein said block copolymer is such that:
the first block is obtained from at least one acrylate monomer of formula CH₂═CH—COOR₂ in which R₂ represents a C₄ to C₁₂ cycloalkyl group, and at least one methacrylate monomer of formula CH₂═C(CH₃)—COOR′₂ in which R′₂ represents a C₄ to C₁₂ cycloalkyl group, and
the second block is obtained from at least one second monomer, such that the homopolymer obtained has a glass transition temperature of less than or equal to 20° C., and an additional monomer of acrylic acid type.

5. The composition according to claim 4, wherein R₂ and R′₂ independently or simultaneously represent an isobornyl group.

6. The composition according to claim 1, wherein said block copolymer comprises from 50% to 80% by weight of isobornyl methacrylate/acrylate, from 10% to 30% by weight of isobutyl acrylate and from 2% to 10% by weight of acrylic acid.

7. The composition according to claim 1, comprising from 0.5% to 30% by weight of block ethylenic copolymer (a), relative to the total weight of the composition.

8. The composition according to claim 1, wherein the hydrocarbon-based non-volatile oil (b) is chosen from isodecyl neopentanoate, isopropyl myristate, isopropyl palmitate, 2-ethylhexyl 2-ethylhexanoate, isononyl isononanoate, isodecyl isononanoate, dicapryl carbonate, dicapryl ether, and mixtures thereof.

9. The composition according to claim 8, wherein in which the hydrocarbon-based non-volatile oil (b) is isodecyl neopentanoate.

10. The composition according to claim 1, wherein hydrocarbon-based non-volatile oil (b) is present in 0.1% to 30% by weight relative to the total weight of said composition.

11. The composition according to claim 1, wherein the volatile oil (c) is a hydrocarbon-based volatile oil.

12. The composition according to claim 1, wherein the volatile oil (c) is present in 1% to 80% by weight relative to the total weight of said composition.

13. The composition according to claim 1, wherein further comprising at least one colorant.

14. The composition according to claim 1, wherein it is a foundation.

15. A process comprising application of the composition of claim 1 to a keratin material.

16. The process according to claim 15, comprising application of the composition to human skin.
